(19) United States
(12) Patent Application Publication
(10) Pub. No.: US 2024/0050478 A1
(43) Pub. Date: Feb. 15, 2024

(54) Method for Culturing Natural Killer Cells Using T Cells

(71) Applicants: GC CELL CORPORATION, Yongin-si (KR); Mogam Institute for Biomedical Research, Yongin-si (KR)

(72) Inventors: Bo Kyung Min, Yongin-si (KR); Hana Choi, Yongin-si (KR); Yu Kyeong Hwang, Yongin-si (KR)

(21) Appl. No.: 18/455,003

(22) Filed: Aug. 24, 2023

Related U.S. Application Data
(62) Division of application No. 17/220,865, filed on Apr. 1, 2021, now Pat. No. 11,766,456.
Application No. 17/220,865 is a continuation of application No. 15/527,752, filed on May 18, 2017, now abandoned.

(30) Foreign Application Priority Data
Nov. 26, 2014 (KR) 10-2014-0166705

(51) Int. Cl. A61K 35/17; C12N 5/0783

(52) U.S. Cl. CPC A61K 35/17; C12N 5/0646; A61K 2035/124

(57) ABSTRACT

The present invention relates to a method for producing natural killer cells using T cells, and more particularly, to a method for producing natural killer cells, which comprises culturing seed cells using CD4 (+) T cells as feeder cells. The method for producing natural killer cells using T cells according to the present invention is a method capable of producing natural killer cells by selectively proliferating only natural killer cells from a small amount of seed cells while maintaining the high killing activity of the natural killer cells. The method of the present invention can produce a large amount of natural killer cells that can be frozen, and thus is useful for commercialization of cell therapeutic agents.

### TECHNICAL FIELD

The present invention relates to a method for producing natural killer cells using T cells, and more particularly, to a method for producing natural killer cells, which comprises culturing seed cells using CD4 (+) T cells as feeder cells.

### BACKGROUND ART

As therapy for preventing the metastasis and recurrence of cancer and extending the survival time of terminal cancer patients, immunotherapy based on the immune function of patients is receiving attention. In recent years, immunotherapy with genetically engineered T cells expressing antigen-specific chimeric antigen receptors (CARs) has been recognized as a promising approach for cancer therapy. However, when T cells of other people are used, they can cause series graft-versus-host disease (GVHD) due to major histocompatibility complex (MHC) restriction. For this reason, for commercialization of cell therapy, natural killer (NK) cells are much more useful, which can be used for allogeneic transplantation and are possible to produce in large amount and to freeze.

Natural killer cells are known as lymphoid cells that account for about 10-15% of peripheral blood lymphocytes and play an important role in innate immune responses. Unlike T cells, natural killer cells recognize their target in an MHC non-restricted manner, and activating receptors (such as NKG2D, NCR (NKp30, NKp44, or NKp46)) in natural killer cells compete with inhibitory receptors such as KIR or CD94/NKG2A to display activity and eliminate tumor target cells. Natural killer cells can exhibit antiviral effects, an-GvH effects, and anticancer effects. Particularly, natural killer cells directly kill malignant tumors, including sarcoma, myeloma, carcinoma, lymphomas and leukemia, or contribute to adaptive immune activation by inducing dendritic cell (DC) activity or tumor-specific cytotoxic T lymphocytes (CTLs), thereby eliminating abnormal cells which are tumor cells or cells developing into tumor cells.

The anticancer effects of natural killer cells were demonstrated through allogeneic hematopoietic stem cell transplantation, and it was found that donor natural killer cells inhibit microtumor remaining after transplantation of T cell-depleted hematopoietic stem cells. In addition, because the graft-versus-tumor (GVT) effect of donor natural killer cells significantly increases when there is KIR (killer cell immunoglobulin-like receptors) -MHC mismatch between the donor and the recipient, the allogeneic natural killer cells is much more effective than the use of the cancer patient's own autologous natural killer cells having reduced function.

Despite the potential of such natural killer cells as therapeutic agents against cancers or infectious diseases, most natural killer cells in the body of normal people exist in a resting state, and natural killer cells in the body of cancer patients lack their function due to the immune escape mechanism of cancer cells. In order to actually use natural killer cells as therapeutic agents, activated natural killer cells capable of recognizing and destroying tumor cells are required, and for this reason, it is very important to activate natural killer cells by ex vivo expansion culture from normal culture or patient's blood. In addition, because the number of natural killer cells in vivo is limited, it is necessarily required to develop a technology capable of producing and freezing natural killer cells in large amounts that can sufficiently exhibit anticancer effects.

In ex vivo expansion culture of natural killer cells, PBMC, CD3- cells, CD3-CD56+ cells, CD56+ cells or the like are used as seed cells, and cytokines such as IL-2, IL-12, IL-15 or IL-21, and OKT-3 antibody (Condiotti et al., Experimental Hematol. 29(1) :104-113, 2001) stimulating LPS (Goodier et al., J. Immunol. 165(1): 139-147, 2000) and CD3, are used as growth factors for natural killer cells. However, the use of the above-mentioned growth factors alone allows natural killer cells to increase about 3-10-fold and cannot achieve sufficient proliferation. For this reason, in several studies, there was an attempt to proliferate natural killer cells using various types of feeder cells. It has been reported that the use of the leukemia cell line CTV-1 showed little or no improvement in proliferation (North et al., J. Immunol. 178(1) :85-94, 2007), and culture with EBV-LCL for 21 days resulted in an increase in cell number of an average of about 490-fold (Berg et al., Cytotherapy, 11(3):341-355, 2009). It has been reported that, when culture was performed for 7 days to 3 weeks using artificial APCs (antigen presenting cells) obtained by expressing 4-1 BBL and membrane-bound IL-15 in the K562 cell line, cell number increased an average of 90-209-fold (Fujisaki et al., Cancer Res. 69(9) : 4010-4017, 2009). In addition, it has been reported that when a K562 cell line having the MICA, 4-1BBL and IL-15 expressed therein was cultured for 3 weeks, cell number increased an average of 350-fold (Gong et al., Tissue Antigens, 76 (6) : 467-475, 2010), and when a K562 cell line having membrane-bound IL-15 expressed therein was cultured for 3 weeks while the cells were re-stimulated at 7-day intervals, cell number increased an average of 21,000-fold (Denman et al., PlosOne, 7(1) : e30264, 2012). In addition, it has been reported that, when PBMCs were cultured for 14 days using KL-1 (human T lymphoblast) and EBV-transformed B cells as feeder cells, an average of a 740-fold increase in the number of natural killer cells was induced (Lim et al., Cancer Res., 73(8) : 2598-6607, 2013).

In Korean Patent No. 10-1133185, the present inventors have disclosed that PBMCs stimulated with OKT-3 can be used as feeder cells, and also disclosed that, when the PBMCs were cultured for 14 days, a 691-fold increase in cell number could be induced (Lim et al., PlosOne, 7(1) : e53611, 2012). In addition, the present inventors have disclosed that, when PBMCs were re-stimulated twice or more with feeder cells, an increase of thousands to tens of thousands of times in cell number could be induced (WO2013/094988). However, in the case of mass culture, the required amounts of PBMCs for use as feeder cells increases and the results of culture of natural killer cells change depending on the characteristics of each donor, and for these reasons, it is actually difficult to achieve the smooth supply and mass culture of raw materials for commercialization and the management of donors.

Accordingly, the present inventors have made extensive efforts to develop seed cells capable of substituting for PBMCs, and as a result, have found that among PBMCs, T lymphocytes, particularly helper T cells (Th cells), are very important in the proliferation of natural killer cells, and T-cell leukemia-lymphoma cell lines which can proliferate while having the characteristics of Th cells can selectively induce the culture of natural killer cells and allow natural killer cells to stably proliferate, thereby completing the present invention.

### DISCLOSURE OF INVENTION

### Technical Problem

It is an object of the present invention to provide a method of producing natural killer cells by culturing and proliferating the cells in an efficient and stable manner. Another object of the present invention is to provide natural killer cells produced by the above method, a composition for preventing or treating cancer or infectious disease, which comprises the natural killer cells as an active ingredient, and a method for preventing or treating cancer or infectious disease, comprising administering to a subject in need thereof a therapeutically effective amount of the above composition.

### Technical Solution

To achieve the above objects, the present invention provides a method for producing natural killer cells, wherein T cells, particularly CD4 (+) T cells, are used as feeder cells that stimulate the natural killer cells.

The present invention also provides natural killer cells produced by the above method.

The present invention also provides a composition for preventing or treating cancer or infectious disease, which comprises the above natural killer cells as an active ingredient.

The present invention also provides a method for preventing or treating cancer or infectious disease, administering to a subject in need thereof a therapeutically effective amount of the above composition.

### BRIEF DESCRIPTION OF THE DRAWINGS

FIG. 1a shows the results of measuring the increase in the number of total nucleated cells and natural killer cells, obtained by culturing PBMC seed cells using various T cells as feeder cells for 14 days (condition 1 in Table 1 below).

FIG. 1b shows the results of measuring the increase in the number of total nucleated cells and natural killer cells, obtained by culturing PBMC seed cells using various T cells as feeder cells, and then re-stimulating the cells with various T cells serving as feeder cells, and culturing the cells up to day 21 (condition 2 in Table 1 below).

FIG. 1c shows the results of measuring the increase in the number of total nucleated cells and natural killer cells, obtained by culturing CD3 (+) cell-depleted PBMC seed cells using various T cells as feeder cells for 14 days (condition 3 in Table 1 below).

FIG. 1d shows the results of measuring the increase in the number of total nucleated cells and natural killer cells, obtained by culturing CD3 (+) cell-depleted PBMC seed cells using PBMC feeder cells, and then re-stimulating the cells with various T cells serving as feeder cells, and culturing the cells up to day 21 (condition 4 in Table 1 below).

FIG. 1e shows the results of measuring the increase in the number of total nucleated cells of poorly proliferating donors, cultured under condition 3 of Table 1 below.

FIG. 2 shows the cell viabilities of natural killer cells cultured using various T cells as feeder cells. (a): cell viability of natural killer cells cultured under condition 1 shown in Table 1; (b): cell viability of natural killer cells cultured under condition 2 shown in Table 1; (c): cell viability of natural killer cells cultured under condition 3 shown in Table 1; (d) : cell viability of natural killer cells cultured under condition 4 shown in Table 1; and (e) cell viability of natural killer cells of poorly proliferating donors, cultured under condition 2 shown in Table 1.

FIG. 3a shows the identity and purity of natural killer cells obtained by culturing PBMC seed cells using various T cells as feeder cells for 14 days (condition 1 in Table 1 below).

FIG. 3b shows the identity and purity of natural killer cells obtained by culturing PBMC seed cells using various T cells as feeder cells, and then re-stimulating the cells with various T cells serving as feeder cells, and culturing the cells up to day 21 (condition 2 in Table 1 below).

FIG. 3c shows the identity and purity of natural killer cells obtained by culturing CD3 (+) cell-depleted PBMC seed cells using various T cells as feeder cells for 14 days (condition 3 in Table 1 below).

FIG. 3d shows the identity and purity of natural killer cells obtained by culturing CD3 (+) cell-depleted PBMC seed cells using PBMC feeder cells, and then re-stimulating the cells with various T cells serving as feeder cells, and culturing the cells up to day 21 (condition 4 in Table 1 below).

FIG. 3e shows the identity and purity of natural killer cells of poorly proliferating donors, obtained by culture under condition 3 of Table 1 below.

FIG. 4a shows an activation marker in natural killer cells obtained by culturing PBMC seed cells using various T cells as seed cells for 14 days (condition 1 in Table 1 below 1).

FIG. 4b shows an activation marker in natural killer cells obtained by CD3 (+) cell-depleted PBMC seed cells using various T cells as feeder cells for 14 days (condition 3 in Table 1 below).

FIG. 5a shows the cell killing activities against various cancers of natural killer cells obtained by culturing PBMC seed cells using various T cells as feeder cells for 14 days (condition 1 in Table 1 below).

FIG. 5b shows the cell killing activities against various cancers of natural killer cells obtained by culturing PBMC seed cells using various T cells as feeder cells, and then re-stimulating the cells with various T cells serving as feeder cells, and culturing the cells up to day 21 (condition 2 in Table 1 below).

FIG. 5c shows the cell killing activities against various cancers of natural killer cells obtained by culturing CD3 (+) cell-depleted PBMC seed cells using various T cells as feeder cells for 14 days (condition 3 in Table 1 below).

FIG. 5d shows the cell killing activities against various cancers of natural killer cells obtained by culturing CD3 (+) cell-depleted PBMC seed cells using PBMC feeder cells, and then re-stimulating the cells with various T cells serving as feeder cells, and culturing the cells up to day 21 (condition 4 in Table 1 below).

FIG. 5e shows the cell killing activities against K562 of natural killer cells of poorly proliferating donors, obtained by culture under condition 3 of Table 1 below.

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods, which will be described below, are those well known and commonly employed in the art.

In the present invention, it was found that, in a process of producing natural killer cells by proliferating the cells using various T cells as feeder cells, T cells, particularly CD4 (+) T cells, induce the selective proliferation of natural killer cells, and thus the proliferation of the natural killer cells increases and the cell killing activity thereof also increases.

In one aspect, the present invention is directed to a method for producing natural killer cells, wherein CD4 (+) T cells are used as feeder cells that stimulate the natural killer cells.

Concretely, the present invention is directed to a method for producing natural killer cells, wherein CD4 (+) T cells isolated ex vivo, CD4 (+) T cells cultured ex vivo, or CD4 (+) T cell lines are used as feeder cells, and one or more types of cells selected from the group consisting of peripheral blood cells, peripheral blood leukocyte cells, peripheral blood mononuclear cells (PBMCs), enriched natural killer cells and isolated natural killer cells are used as seed cells.

The method for producing natural killer cells according to the present invention may concretely comprise the following steps, but is not limited thereto:
(i) a step of isolating peripheral blood leukocyte cells, peripheral blood mononuclear cells (PBMCs), T cell-depleted mononuclear cells or natural killer cells from human peripheral blood;
(ii) a step of culturing natural killer cells in a medium containing an interleukin protein and a T cell-stimulating antibody having a low affinity for T cells, in the presence of inactivated or non-inactivated T cells;
(iii) a step of re-stimulating the natural killer cells with T cells in a medium containing an interleukin protein and a T cell-stimulating antibody having a low affinity for T cells, and further culturing the natural killer cells.

Hereinafter, the present invention will be described in further detail.

Natural killer (NK) cells are present in an amount of about 10-15% in the blood of normal people, and have high killing ability when they react with non-self material. Natural killer cells non-specifically and immediately act in response to the infection of cells with various viruses, the penetration of bacteria or the production of abnormal cells to thereby remove foreign matter. However, the number of natural killer cells present in the body is not so large, and the number of effective natural killer cells required to exhibit therapeutic effects should be very large. For this reason, a method for the proliferation and production of effective natural killer cells is required.

Methods for proliferating natural killer cells include a method comprising isolating natural killer cells only, suitably stimulating the isolated natural killer cells with feeder cells, and proliferating the stimulated natural killer cells, and a method comprising selectively proliferating natural killer cells from peripheral blood lymphocytes (PBLs) or peripheral blood mononuclear cells (PBMCs) to thereby obtain a relatively large amount of natural killer cells. To isolate natural killer cells from peripheral blood, a conventional method known to one skilled in the art may be used, and commercially available natural killer cells may also be purchased and used. PBMCs are separated into lymphocytes and monocytes, and the lymphocytes are further divided into T cells, B cells, and natural killer cells. Among them, the T cells are further divided into helper T cells (Th cells) and cytotoxic T cells (Tc cells). In the method of culturing natural killer cells using PBMC feeder cells, the mass proliferation of T cells is induced, and the proliferation of natural killer cells is limited, and for this reason, an expensive complex step of removing T cells is required before or after culture. Accordingly, in the present invention, whether any cell group among the PBMC groups contributes to the proliferation of natural killer cells was examined. As a result, it was found that T cells contribute to the proliferation of natural killer cells, and among them, Th cells are very important in the proliferation of natural killer cells. Based on this finding, T cells that can proliferate while having the characteristics of Th cells were used as feeder cells capable of PBMCs in the culture of natural killer cells.

As used herein, the term “feeder cells” refers to cells that produce various metabolites due to their metabolic activity to thereby assist in the proliferation of target cells, even though these cells cannot proliferate.

Feeder cells that are used in the present invention may be CD4 (+) T cells isolated ex vivo, CD4 (+) T cells expansion-cultured ex vivo, or a CD4 (+) T cell line (T lymphoma cell line). The CD4 (+) T cell line (T lymphoma cell line) that is used in the present invention is concretely H9, HuT78, Loucy, Molt3, Molt-13, PEER, RPMI8402, or TALL-01, more concretely H9 or HuT78, but is not limited thereto.

T cells that are used as the feeder cells may be inactivated or non-inactivated cells whose proliferation was inhibited. Concretely, the T cells may be inactivated to ensure their safety. As a method for inactivating the T cells, a conventional method known in the art may be used, and for example, a method of irradiating the T cells with gamma-rays may be used. If non-inactivated T cells are used, they can be killed by natural killer cells during culture, because they are mostly tumor cells.

The proliferation method as described in the present invention uses T cells as feeder cells. This method has an advantage in that, because the culture of natural killer cells is selectively induced in seed cells such as PBMCs without removing T cells, the culture can be stably performed without a significant difference in culture results between donors. Thus, it is possible to obtain an increased amount of natural killer cells for therapeutic purposes in an efficient and stable manner.

As used herein, the term “seed cells” means cells capable of proliferating to natural killer cells by suitable culture. Concretely, the seed cells that are used in the present invention may be one or more types selected from the group consisting of peripheral blood cells, peripheral blood leukocytes, PBMCs (peripheral blood mononuclear cells), enriched natural killer cells, and isolated natural killer cells, but are not limited thereto. More concretely, the seed cells may be CD3 (+) cell-depleted cells (CD3 (−) cells).

In addition, in the method for producing natural killer cells according to the present invention, natural killer cells are concretely cultured in a medium containing an interleukin protein and a T cell-stimulating antibody having a low affinity for T cells, but the scope of the present invention is not limited thereto. Herein, the T cell-stimulating antibody having a low affinity for T cells is a protein that reacts specifically with CD3 antigen, a group of molecules which associate with a T cell receptor (TCR) to form an antigen recognition complex, in which the CD3 molecule has a longer intracellular domain compared to TCR and functions to transfer an antigen recognition signal into cells. The T cell-stimulating antibody having a low affinity for T cells, which can be used in the present invention, is concretely anti-CD3 antibody, more concretely OKT-3, UCHT1, HIT3a or the like, most concretely OKT-3 antibody.

As used herein, the term “interleukin (IL) protein” refers to a collection of biologically active proteins produced by immune cells such as lymphocytes, monocytes or macrophages, and means a group of molecular species in cytokines. Examples of an interleukin protein, which may be used in the present invention, include IL-2, IL-15, IL-12, IL-18, IL-21 and the like. Concretely, the interleukin protein is IL-2 protein.

The method for producing natural killer cells according to the present invention concretely comprises: adding natural killer cells and a T lymphoma cell line to a conventional animal cell culture medium such as AIM-V medium, RIMI 1640, CellGro SCGM, X-VIVO20, IMDM or DMEM; adding an interleukin protein and T cell-stimulating antibody having a low affinity for T cells to the culture medium; and culturing the cells, but is not limited thereto. In an example of the present invention, cells were cultured in a medium containing OKT-3 antibody and IL-2. In the present invention, OKT-3 antibody is used at a concentration of 0.1-100 ng/ml, concretely about 10 ng/ml, and IL-2 is used at a concentration of 10-2000 U/ml, concretely about 500 U/ml. In addition, the culture medium may further contain serum or plasma and an additional growth factor that supports the proliferation of lymphocytes. The type of serum or plasma that is added to the medium is not specifically limited, and thus various types of commercially available serum or plasma may be used in the present invention. Concretely, autologous human serum or plasma is used in the present invention.

The method for producing natural killer cells according to the present invention comprises a step of culturing natural killer cells in a medium containing an interleukin protein and a T cell-stimulating antibody having a low affinity for T cells, in the presence of T cells as feeder cells that stimulate natural killer cells. For use in the culturing step, the feeder cells and the seed cells are concretely mixed at a ratio of at least 1:1, more concretely 2:1-20:1, most concretely 5:1, but are not limited thereto. Herein, the term “ratio” refers to a ratio based on cell number .

In addition, in the method for producing natural killer cells according to the present invention, the stimulation and culture of natural killer cells with CD4 (+) T cells serving as feeder cells may be repeatedly performed in order to obtain an increased number of natural killer cells. Thus, the method of the present invention may further comprise re-stimulating natural killer cells at a feeder cell-to-seed cell ratio of at least 1:1, concretely 2:1-20:1, most concretely 5:1. Herein, the term “ratio” refers to a ratio based on cell number.

The CD4 (+) T cells that are used in the re-stimulation are concretely H9 or HuT78 cells, but are not limited thereto. In addition, inactivated cells whose proliferation was inhibited, or non-inactivated cells, may be used in the re-stimulation.

In addition, the re-stimulation is concretely performed in a medium containing an interleukin protein and a T cell-stimulating antibody having a low affinity for T cells, at intervals of 5-12 days, more concretely 7 days, but is not limited, and may be repeated at least once. The method of the present invention may further comprise, after the step of culturing the cells in the medium containing the interleukin protein and the T cell-stimulating antibody having a low affinity for T cells, a step of culturing the cells in a medium free of the antibody.

Herein, the T cell-stimulating antibody having a low affinity for T cells may be an anti-CD3 antibody, concretely at least one selected from the group consisting of OKT3, UCHT1 and HIT3a antibodies, most concretely OKT3 antibody, and the interleukin protein may be at least one selected from the group consisting of IL-2, IL-12, IL-15, IL-18, and IL-21, concretely IL-2.

As used herein, the term “stimulating” means inducing the proliferation of natural killer cells by adding feeder cells or the like. In the stimulation step, a T cell-stimulating antibody having a low affinity for T cells may also be used. As used herein, the term “re-stimulating” means re-inducing the proliferation of natural killer cells by adding feeder cells and/or a T cell-stimulating antibody, which has a low affinity for T cells, to the medium after a certain time of culture.

In the method of the present invention, the natural killer cells are cultured for at least 5 days, concretely 5-60 days, more concretely 14-21 days, but are not limited thereto.

The stimulation may start at day 0 of culture and may be repeated at intervals of 5-12 days, concretely 7 days, but is not limited thereto. The cells may be harvested at 5 days or more, concretely 14 days, after the final stimulation, but are not limited thereto.

Natural killer cells cultured according to the method of the present invention as described above can be frozen and are not functionally impaired even when they are thawed. In addition, these cells express higher levels of activating receptors such as NKp44 and NKp46 compared to conventional cells cultured using PBMCs as feeder cells, and thus have increased abilities to kill tumor cells and to secrete cytokines, indicating that these cells can exhibit excellent anticancer effects. Thus, according to the present invention, a cell therapeutic agent effective for tumor treatment can be prepared using a large amount of activated natural killer cells that can be clinically applied.

### EXAMPLES

Hereinafter, the present invention will be described in further detail with reference to examples. It will be obvious to a person having ordinary skill in the art that these examples are illustrative purposes only and are not to be construed to limit the scope of the present invention. Thus, the substantial scope of the present invention will be defined by the appended claims and equivalents thereof.

### Example 1: Preparation of PBMCs and Various T Cells

1-1: Preparation of PBMC Seed Cells and CD3 (+)-Depleted Seed Cells

Peripheral blood mononuclear cells (PBMCs) were dispensed into vials and frozen in liquid nitrogen. One frozen PBMC vial was thawed and transferred into a 50 mL tube, and the cells were suspended in 20 mL of PBS (phosphate buffered saline) containing 1 vol % FBS (fetal serum bovine) or autoplasma and were centrifuged at 1200 rpm at 4° C. for 10 minutes. The PBMC pellets were suspended in 10 mL of MACS running buffer, and the cells were counted using an Adam cell counter system.

1×10⁷ PBMC seed cells were dispensed into each of 50 mL tubes. To obtain PBMC feeder cells and CD3 (+) cell-depleted seed cells, 5×10⁷ cells were transferred into each of 50 mL fresh tubes and centrifuged at 1200 rpm at 4° C. for 10 minutes.

In the case of PBMC feeder cells, the cell pellets were suspended in 10 mL of 1 vol % autoplasma-containing CellGro medium (Cellgenix).

To obtain CD3 (+)-depleted seed cells, 400 μL of MACS running buffer and 100 μL of CD3 magnetic beads (Miltenyi biotech) were added to 5×10⁷ PBMC cell pellets which were then incubated at 4° C. for 20 minutes. The resulting cells were washed with 10-20 mL of MACS running buffer, and then centrifuged at 1200 rpm at 4° C. for 10 minutes and suspended aging in 2 mL of MACS running buffer. The cells were separated using a CS column (Miltenyi Biotech, 130-041-305) equipped with VarioMACS (Miltenyi Biotech), and the column was washed to reach a final volume of 20 mL, thereby recovering the cells. The cells were counted using an Adam cell counter system, and 1×10⁷ cells were dispensed into 50 mL fresh tubes and centrifuged at 1200 rpm at 4° C. for 10 minutes. The cell pellets were suspended in 10 mL of CellGro medium (Cellgenix) containing 1 vol % autoplasma.

### 1-2: PBMC Feeder Cells and Various T Feeder Cells

5×10⁷ PBMC feeder cell pellets separated in Example 1-1 were suspended in 10 mL of 1 vol % autoplasma-containing CellGro medium (Cellgenix) and irradiated with 2000 cGy of gamma rays in a gamma-ray irradiator, thereby preparing PBMC feeder cells.

Various T cells were recovered from culture flasks and centrifuged at 1200 rpm at 4° C. for 10 minutes, and then 5×10⁷ cells were dispensed into each tube and centrifuged at 1200 The T cells were suspended in 10 rpm at 4° C. for 10 minutes. mL of 1 vol % autoplasma-containing CellGro medium (Cellgenix), and then irradiated with 15000-30000 cGy of gamma rays in a gamma ray irradiator, thereby preparing T cells for use as feeder cells.

### Example 2: Culture of Natural Killer Cells Using Various T Cells as Feeder Cells

Conditions for culturing PBMC seed cells and CD3 (+)-depleted seed cells using T cells as feeder cells are shown in Table 1 below.

TABLE 1

| | Seed | Feeder cells | | Culture |
|---|---|---|---|---|
| | cells | D 0 stimulation | D 7 stimulation | period |
| Condition 1 | PBMC | PBMC & T | None | 14 days |
| | | lymphoma | | |
| Condition 2 | PBMC | PBMC & T | PBMC & T | 21 days |
| | | lymphoma | lymphoma | |
| Condition 3 | CD3(−) | PBMC & T | None | 14 days |
| | | lymphoma | | |
| Condition 4 | CD3(−) | PBMC | PBMC & T | 21 days |
| | | | lymphoma | |

### 2-1: 14-Day Culture Conditions

For culture of natural killer cells, 500 IU of IL-2 and 10 ng/ml of OKT-3 were added to a culture vessel (a flask or a cell culture bag). At day 0 of culture, PBMC seed cells and PBMC feeder cells or T feeder cells were added to the culture vessel at a ratio of 1:5 in an amount of 0.5-10 mL for each cell type, and 0.5-10 mL of 1 vol % autoplasma-containing CellGro medium (Cellgenix) was added to the cells which were then stationary-cultured in an incubator at 37° C. for 3-5 days (condition 1). In addition, at day 0 of culture, CD3 (−) seed cells and PBMC feeder cells or T seed cells were added to a culture vessel at a ratio of 1:5 in an amount of 0.5-10 mL for each cell type, and 0.5-10 mL of 1 vol % autoplasma-containing CellGro medium (Cellgenix) was added to the cells which were then stationary-cultured in an incubator at 37° C. for 3-5 days (condition 3).

In the case of conditions 1 and 3 shown in Table 1, the cells were counted at days 3-5 of culture, and diluted to a density of about 2-5×10⁵ cells/mL with CellGro medium (Cellgenix) containing 500 IU of IL-2 (Proleukin) and 1 vol % autoplasma. The diluted cells were stationary-cultured again in a suitable culture vessel. Next, the cells were suspension-cultured up to day 14 while the cells were counted at intervals of 2-3 days and diluted to a density of 5-10×10⁵ cells/mL CellGro medium (Cellgenix) containing 500 IU of IL-2 and 1 vol % autoplasma. At day 14 of suspension culture, natural killer cells were harvested.

The proliferation rates of the natural killer cells cultured under condition 1 were compared, and as a result, it was shown that PBMC feeder cells showed a 147-fold increase in the number of total nucleated cells (TNCs), and H9 feeder cells and HuT78 feeder cells showed a 298-fold increase and a 485-fold increase, respectively, which were significantly higher than the fold increase shown by PBMC feeder cells.

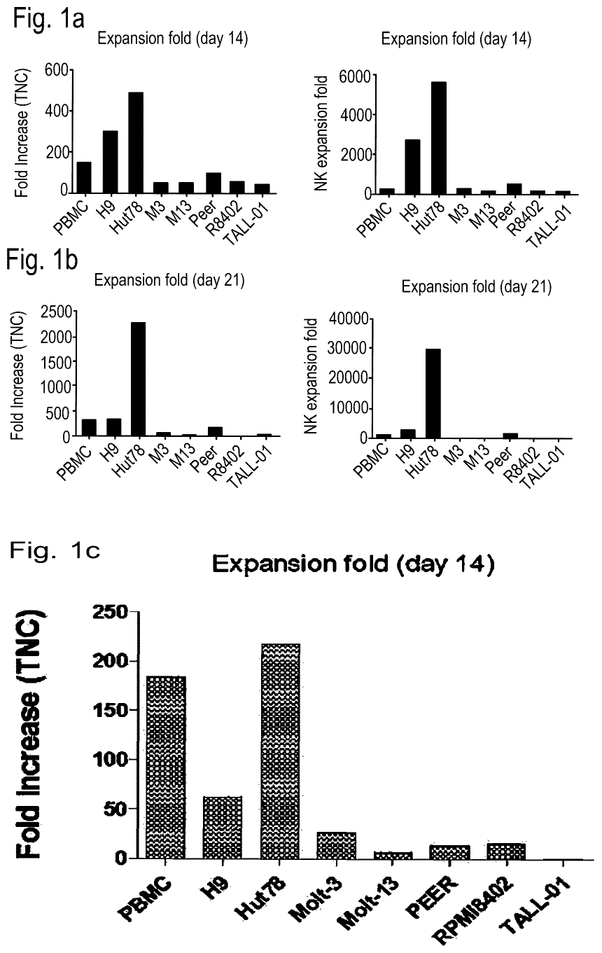

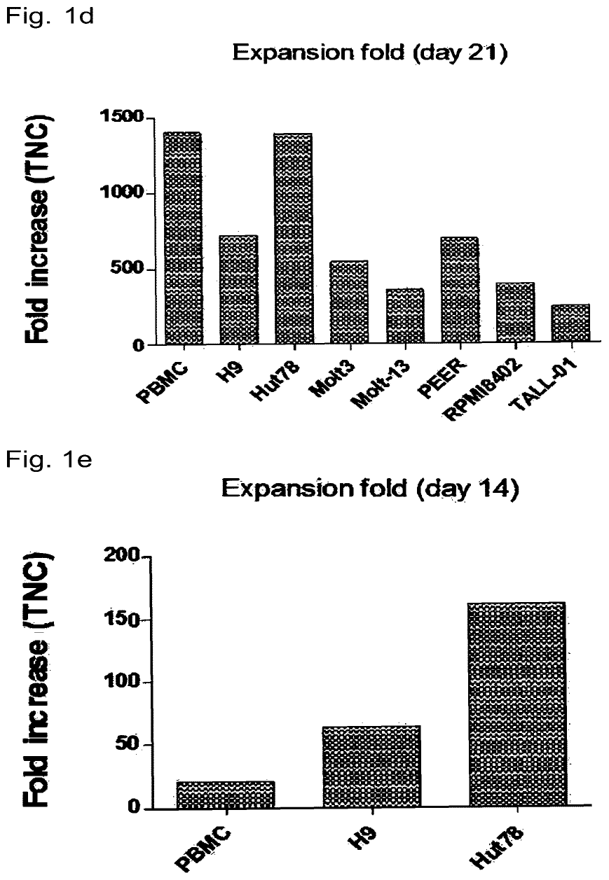

Also, in the proliferation rate of natural killer cells among total nucleated cells, PBMC feeder cells showed a 247-fold increase in the number of natural killer cells, H9 feeder cells showed a 2752-fold increase which was about 10 times higher than that shown by PBMC feeder cells, and HuT78 feeder cells showed a 5649-fold increase which was about 20 times higher than that shown by PBMC feeder cells. In addition, other T cells showed proliferation rates lower than that shown by PBMCs (see FIG. 1a).

In addition, the proliferation rates of natural killer cells cultured under condition 3 were compared, and as a result, PBMCs showed a 184-fold increase in the number of total nucleated cells, H9 feeder cells showed a 62-fold increase, and HuT78 feeder cells showed a 217-fold increase (see FIG. 1c). Other T cells all showed proliferation rates lower than that shown by H9.

### 2-2: Re-stimulation Conditions in 21-Day Culture

For culture of natural killer cells, 500 IU of IL-2 and 10 ng/ml of OKT-3 were added to a culture vessel (a flask or At day 0 of culture, PBMC seed cells a cell culture bag). and PBMC feeder cells or T feeder cells were added to the culture vessel at a ratio of 1:5 in an amount of 0.5-10 mL for each cell type, and 0.5-10 mL of 1 vol % autoplasma-containing CellGro medium (Cellgenix) was added to the cells which were then stationary-cultured in an incubator at 37° C. for 3-5 days (condition 1). For re-stimulation at day 7 of culture, natural killer cells, stimulated and cultured with PBMC feeder cells at day 0, were re-stimulated with PBMC feeder cells and various T feeder cells, and cells, stimulated and cultured with T feeder cells at day 0, were re-stimulated using the same cells as feeder cells (condition 2). In addition, at day 0 of culture, CD3 (−) seed cells and PBMC feeder cells were added to a culture vessel at a ratio of 1:5 in an amount of 0.5-10 mL for each cell type, and 0.5-10 mL of 1 vol % autoplasma-containing CellGro medium was added to the cells which were then stationary-cultured in an incubator at 37° C. for 3-5 days, thereby achieving the first stimulation of natural killer cells. In this case, for re-stimulation at day 7 of culture, natural killer cells were re-stimulated with PBMC feeder cells and various T feeder cells and cultured for 21 days (condition 4).

For re-stimulation at day 7 of culture, the natural killer cells being cultured were counted and diluted to a density of 2-5×10⁵ cells/mL with 1 vol % autoplasma-containing CellGro medium (Cellgenix), and PBMC and T feeder cells were diluted 3- to 10-fold and suspended in 1 vol % autoplasma-containing CellGro medium (Cellgenix), and then inactivated by irradiation with 2000 and 15000-30000 cGy of gamma-rays, respectively, in a gamma-ray irradiator. 500 IU of IL-2 and 10 ng/ml of OKT-3 were added to the medium, and the prepared two types of cells were co-cultured. Next, the cells were suspension-cultured up to day 21 while the cells were counted at intervals of 2-3 days and diluted to a density of 5-10×10⁵ cells/mL with CellGro medium (Cellgenix) containing 500 IU of IL-2 and 1 vol % autoplasma. At day 21 of suspension culture, natural killer cells were harvested.

The proliferation rates of natural killer cells cultured under condition 2 shown in Table 1 were compared. As a result, it was shown that the fold increase in the number of total nucleated cells was higher in the order of PBMCs (334-fold), H9 (358-fold) and HuT78 (2282-fold). The fold increase in the number of natural killer cells was 1257-fold for PBMCs, 2677-fold for H9, and 29455-fold for HuT78 (FIG. 1b).

In addition, the proliferation rates of natural killer cells cultured under condition 4 of Table 1 while performing first stimulation with PBMC feeder cells second and stimulation with PBMC feeder cells and each type of T feeder cells were compared. As a result, it was shown that the fold increase in the number of total nucleated cells was 1402-fold for PBMCs, 720-fold for H9, and 1393-fold for HuT78 (FIG. 1d). Other T cells showed an increase in cell number of about 500-fold.

As described above, it could be seen that, in 14-day culture and 21-day culture, H9 and HuT78 can be used as feeder cells that are very excellent in terms of proliferation rate compared to PBMCs.

The results of evaluation performed using CD3 (-) cells as seed cells and various T cells as feeder cells did slightly differ from the results obtained using PBMCs as feeder cells. Specifically, the proliferation rate of natural killer cells was similar between the PBMC feeder cells and the HuT78 feeder cells, and H9 feeder cells showed a reduced proliferation rate of natural killer cells compared to the PBMC feeder cells.

### 2-3: Culture of Poorly Proliferating Donor Cells

Donor cells that showed poor proliferation when cultured with PBMC feeder cells under condition 4 of Table 1 were selected and used to compare the proliferation rates of natural killer cells.

As a result, it was shown that culture with PBMC feeder cells showed an average of a 21-fold increase in the number of natural killer cells, but H9 and HuT78 showed a 64-fold increase and a 161-fold increase, respectively. Particularly, HuT78 showed an increase in cell number, which was about 8 times higher than that shown by PBMC feeder cells (FIG. 1e). Thus, it could be seen that T feeder cells can overcome the difference between donors with respect to the proliferation of natural killer cells, unlike PBMC feeder cells.

In the case of PBMC feeder cells, a process of depleting T cells from seed cells is necessarily required, because the proliferation of T cells rather than natural killer cells is induced when T cells are present in seed cells. In addition, a preculture process for previously selecting donors is required, because the difference in proliferation rate between donors is significant. However, it was found that T cells, particularly CD4-expressing T cells, could induce the selective proliferation of natural killer cells regardless of the presence or absence of T cells in seed cells and that the proliferation of poorly proliferating donor cells could also be easily induced when culture with PBMC feeder cells was performed.

### Example 3: In Vitro Cell Viability

Among cell counter systems employing a PI dye capable of binding to the intracellular nucleus, an ADAM cell counter system was used to comparatively evaluate in vitro cell viabilities. The number of viable cells was determined by subtracting the measured dead cell count from the measured total cell count, and then cell viability was calculated using the following equation.

Cell viability (%)=(viable cell count/total cell count)×100

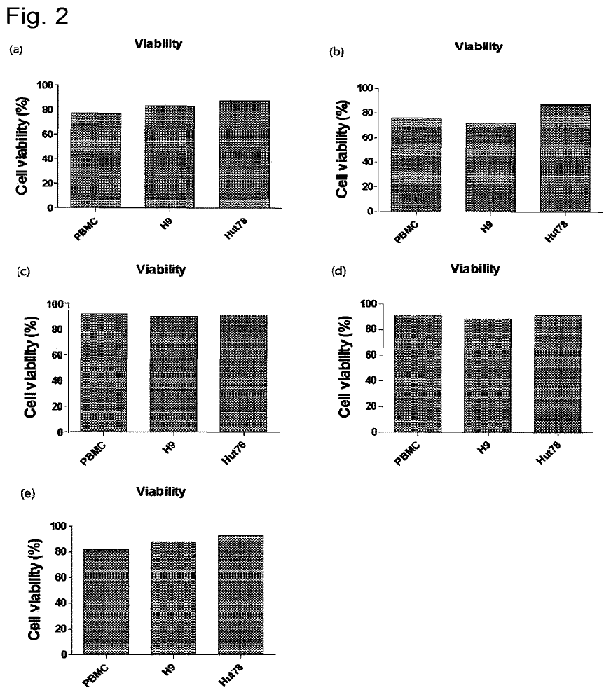

The viability of natural killer cells was measured after culture under conditions 1 and 2 (that is, 14-day culture and 21-day culture of PBMC seed cells with various feeder cells) shown in Table 1. As a result, it was shown that the use of PBMC feeder cells showed a viability lower than 80%, and H9 and HuT78 showed a viability of 80% or higher (FIGS. 2a and 2b).

The viability of natural killer cells was measured after culture under conditions 3 and 4 (that is, 14-day culture and 21-day culture of CD3 (+) T cell-depleted seed cells with various T feeder cells) shown in Table 1 were measured. As a result, it was shown that a high viability of about 90% or higher was shown in all the conditions. In the case of poorly proliferating donor cells, PBMC feeder cells showed a viability of 82%, and HuT78 showed a viability of 93% which was at least 10% higher than that shown by PBMC feeder cells (FIGS. 2c and 2d).

The viability of the cells cultured using PBMCs and various T feeder cells for 14 days or 21 days was generally higher in CD3 (−) seed cells than in PBMC seed cells, and showed no significant difference between the types of seed cells. However, in the case of poorly proliferating donor cells, HuT78 showed a viability which was about 10% higher than that shown by PBMC feeder cells.

Thus, it could be seen that HuT78 cells are feeder cells for culturing natural killer cells, which are useful in terms of cell viability compared to PBMCs.

### Example 4: In Vitro Analysis of Cell Phenotypes

Natural killer cells cultured according to the methods of Examples 1 and 2 were collected before and after culture and centrifuged at 1200 rpm for 5 minutes, and the medium was removed by suction. The cells were diluted with 1 mL of FACS buffer (2.5% FBS-containing PBS), counted, and then diluted to a density of 5×10⁶ cells/mL with FACS buffer. 100 L of the diluted cell solution was dispensed into each of 5 mL FACS tubes (Falcon, 352052), and the phenotypes of the cells were analyzed using the following antibody:
Tube 1: anti-human CD3-FITC (BD Pharmingen, 555332), anti-human CD16-PE (BD Pharmingen, 555407), anti-human CD56-PE-Cy5 (BD Pharmingen, 555517)
Tube 2: anti-human CD14-FITC (BD Pharmingen, 555397), anti-human CD19-PE (BD Pharmingen, 555413), anti-human CD3-PE-Cy5 (BD Pharmingen, 555341)
Tube 3: anti-human CD3-FITC, anti-human NKG2A-PE (R&D system, FAB1059P), anti-human CD56-PE-Cy5
Tube 4: anti-human CD3-FITC, anti-human NKG2C-PE (R&D system, FAB138P), anti-humanCD56-PE-Cy5
Tube 5: anti-human CD3-FITC, anti-human NKG2D-PE (R&D system, FAB139P), anti-human CD56-PE-Cy5
Tube 6: anti-human CD3-FITC, anti-human NKp30-PE (BD Pharmingen, 558407), anti-human CD56-PE-Cy5
Tube 7: anti-human CD3-FITC, anti-human NKp44-PE (BD Pharmingen, 558563), anti-humanCD56-PE-Cy5
Tube 8: anti-human CD3-FITC, anti-human NKp46-PE (BD Pharmingen, 557991), anti-human CD56-PE-Cy5
Tube 9: anti-human CD3-FITC, anti-human DNAM-1-PE (BD Pharmingen, 559789), anti-humanCD56-PE-Cy5
Tube 10: anti-human CD3-FITC, anti-human CD25-PE (BD Pharmingen, 555432), anti-human CD56-PE-Cy5
Tube 11: anti-human CD3-FITC, anti-human CD62L-PE (eBioscience, 12-0629-42), anti-human CD56-PE-Cy5
Tube 12: anti-human CD3-FITC, anti-human CD69-PE (R&D systems, FAB23591P), anti-human CD56-PE-Cy5
Tube 13: anti-human CD3-FITC, anti-human CXCR3-PE (BD Pharmingen, 557185), anti-human CD56-PE-Cy5
Tube 14: anti-human CD3-FITC, anti-human CD57-PE (BD Pharmingen, 560844), anti-human CD56-PE-Cy5
Tube 15: anti-human CD3-FITC, PE mouse IgG1 k isotype control (BD Pharmingen, 555749), anti-human CD56-PE-Cy5
Tube 16: FITC mouse IgG1 k isotype control (BD Pharmingen, 555748), PE mouse IgG1 k isotype control (BD Pharmingen, 555749), PE-Cy5 mouse IgG1 k isotype control (BD Pharmingen)

The tubes were stained at a cold temperature for 30 minutes, and the stained cells were added to 2 mL of FACS buffer and centrifuged at 1500 rpm for 5 minutes. The supernatant was removed, and the remaining cells were added again to 2 mL of FACS buffer and centrifuged at 1500 rpm for 5 minutes. Next, the supernatant was removed, and the remaining cells were suspended in 300 μL of FACS buffer, and the phenotypes thereof were analyzed using FACS LSRII Fortessa (Becton Dickinson), thereby determining the identity and purity of the cells. The content was expressed as the contents of CD3(−) CD56 (+) cells and CD16 (+) CD56 (+) cells, and the purity was measured with CD3 (+) for T cells, CD14 (+) for monocytes, and CD19 (+) for B cells.

### 4-1: Cell Identity and Purity

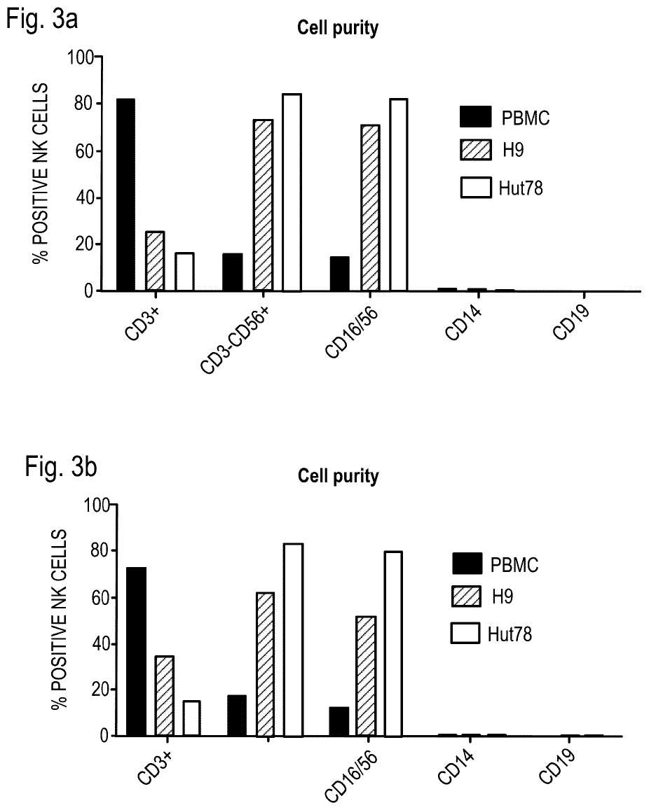

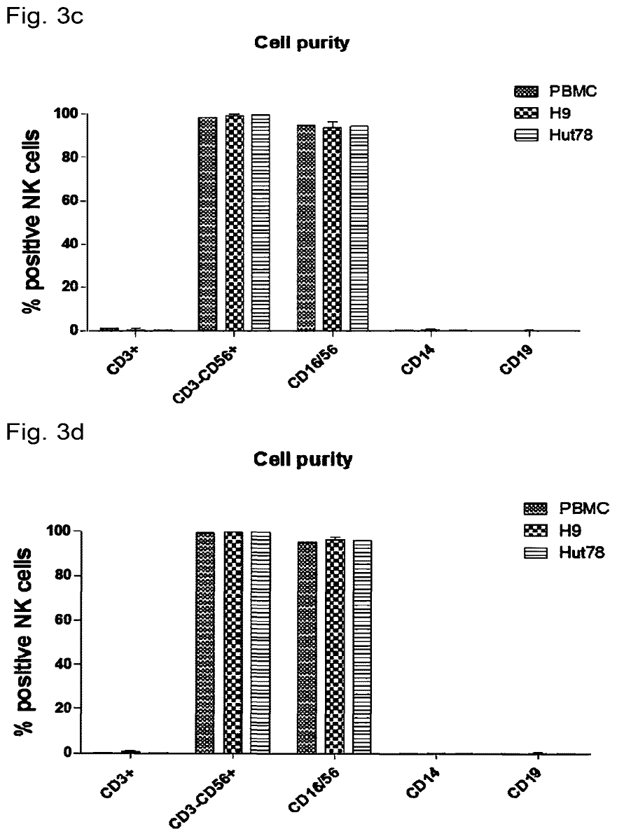

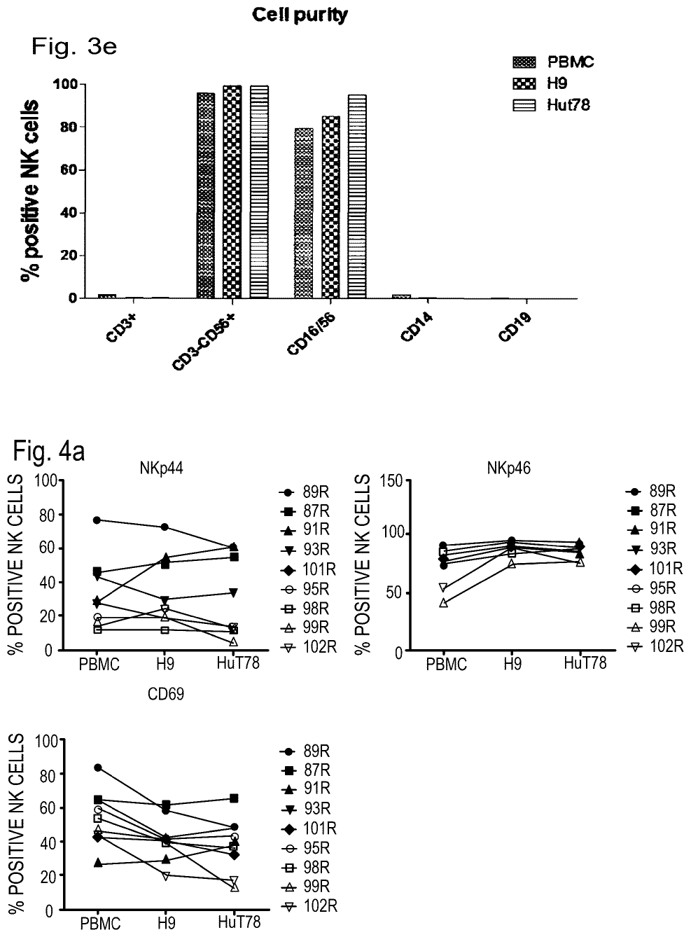

The identity and purity of natural killer cells cultured under conditions 1 and 2 shown in Table 1 were analyzed. As a result, the content of natural killer cells, evaluated after culturing PBMC seed cells with various feeder cells for 14 days, was 15.9% for PBMCs, 73.3% for H9, and 83.3% for HuT78, and the content of natural killer cells, evaluated after 21 days of culture, was 17.4% for PBMCs, 61.1% for H9, and 83.5% for HuT78 (see FIGS. 3a and 3b). Thus, it could be seen that, when PBMCs containing T cells were used as seed cells, in the case of PBMC feeder cells, T cells proliferated 80% or more, whereas in the case of H9 and HuT78, natural killer cells other than T cells selectively proliferated. Particularly, HuT78 showed a high natural killer cell content of 80% or more.

The identity and purity of natural killer cells cultured under conditions 3 and 4 shown in Table 1 were analyzed. As a result, it was shown that, because T cell-depleted CD3 (-) seed cells were used under both the conditions, all the types of feeder cells showed a high natural killer cell content of 95% or more, and the contents of T cells, monocytes and B cells were all measured to be 1% or less (FIGS. 3c and 3d).

In addition, in the case of poorly proliferating donor cells cultured under condition 3 shown in Table 1, it was shown that culture with PBMC feeder cells showed a decrease in purity of about 4% and a decrease in CD16 expression of about 16%, compared to culture with HuT78 (FIG. 3e). Thus, it could be seen that CD4-expressing T cells could induce the selective proliferation of natural killer cells only and can significantly overcome the difference in culture of natural killer cells between donors.

### 4-2: Cell Expression Markers

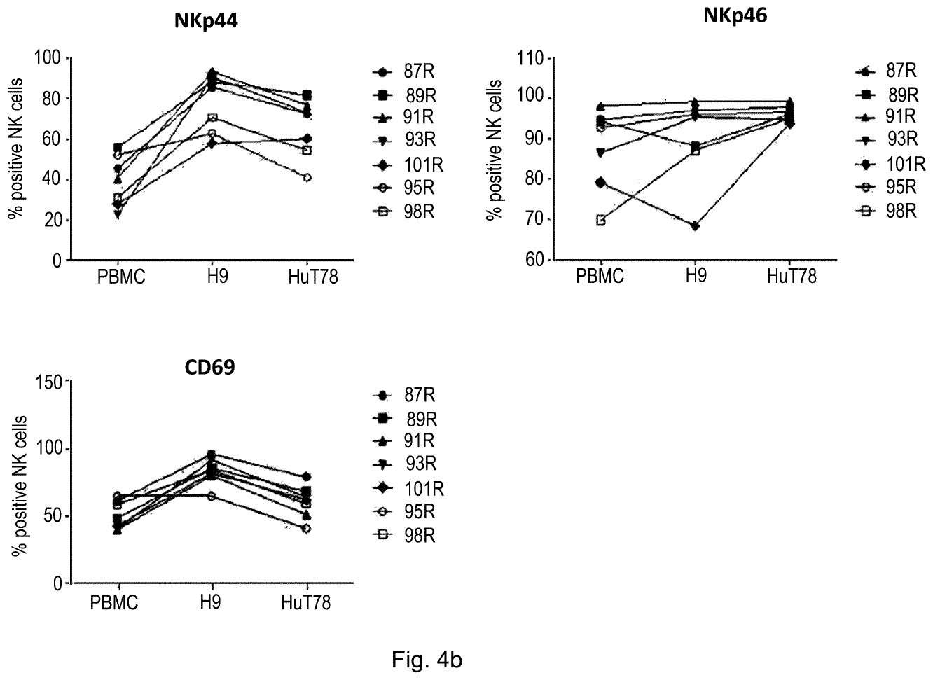

In addition to the identity and purity of cells, the expression of typical natural killer cell receptors, which differs depending on the type of feeder cells, was analyzed. The cell phenotypes of natural killer cells cultured under condition 1 of Table 1 were analyzed. As a result, for NKp44, NKp46 and CD69, the difference in phenotype between culture conditions was observed (FIG. 4a).

In addition, the cell phenotypes of natural killer cells cultured under condition 3 of Table 1 were analyzed. As a result, it was shown that the expression of NKp44 and NKp46 was higher in culture with H9 and HuT78 than in culture with PBMC and that the expression of CD69 was similar between culture with PBMC and culture with HuT78, but increased in culture with H9 (FIG. 4b).

Thus, it could be seen that, although the expression of cell phenotype markers did differ depending on the type of seed cells, the expression of natural killing cell activation markers such as NKp44 and NKp46 was significantly higher in culture with T feeder cells than in culture with PBMC feeder cells. These markers are important factors in the activity of natural killer cells, and the above results indicate that the efficacy of natural killer cells is higher in culture with T cells than in culture with PBMC feeder cells.

### Example 5: In Vitro Cell Killing Activity Against

Various Tumor Cell Lines 1×10⁶ cells of each of target tumor cell lines (K562, HuT78, HuH-7, etc.) were dispensed into each of 15 mL tubes and centrifuged. Then, the cell pellets were suspended in 1 mL of RPMI 1640-10% FBS medium, after which 30 μL of 1 mM Calcein-AM (Molecular probe, C34852) was added thereto, and then the cells were stained in an incubator at 37° C. for 1 hour while light was blocked with silver paper. The tumor cell line stained with Calcein-AM was washed with 10-15 mL of RPMI 1640-10% FBS medium and centrifuged, and the pellets were suspended in 10 mL of RPMI medium to a concentration of 1×10⁵ cells/mL.

3×10⁶ natural killer cells were dispensed into each of 15 mL tubes and centrifuged, and the pellets were suspended in RPMI 1640-10% FBS medium at a desired ratio relative to the target tumor cell line. 100 μL of each of the prepared target tumor cell line and natural killer cell line was dispensed into each well of a round-bottom 96-well plate, and each well was prepared in triplicate. To each spontaneous release well, 100 μL of the stained tumor cell line and 100 μL of RPMI 1640-10% FBS medium were added. To each maximum release well, 100 μL of the stained tumor cell line and 100 μL of 2% Triton-X 100 solution were added. To correct an autofluorescence value present in RPMI 1640-10% FBS medium and 2% Triton-X 100 solution, 200 μL of RPMI 1640-10% FBS medium was added to prepare a medium value, 100 μL of 2% Triton-X 100 solution was added to 100 μL of RPMI 1640-10% FBS medium to prepare the value of the mixture of the two solutions. The difference (A) obtained by subtracting the value of the mixture from the medium value was added to the maximum release value, thereby correcting the autofluorescence value.

The cells were incubated in an incubator at 37° C. for 4 hours under a light-shielding condition, and then the plate was centrifuged at 2000 rpm for 3 minutes. 100 μL of the supernatant was added to each well of a 96-well black plate, and the fluorescence value (OD_{480/535 nm}) was measured using a fluorescence plate reader (Perkin Elmer, VICTOR X3). Based on the measured fluorescence value, the tumor cell killing activity of the natural killer cells was calculated using the following equation:

% of killing=(Average fluorescence value of sample wells−average fluorescence value of spontaneous wells/{(average fluorescence value of maximum wells+A)−average fluorescence value of spontaneous wells}×100

Natural killer cells cultured with various feeder cells were allowed to react with various tumor cell lines, and the direct tumor cell killing activity of the natural killer cells was measured.

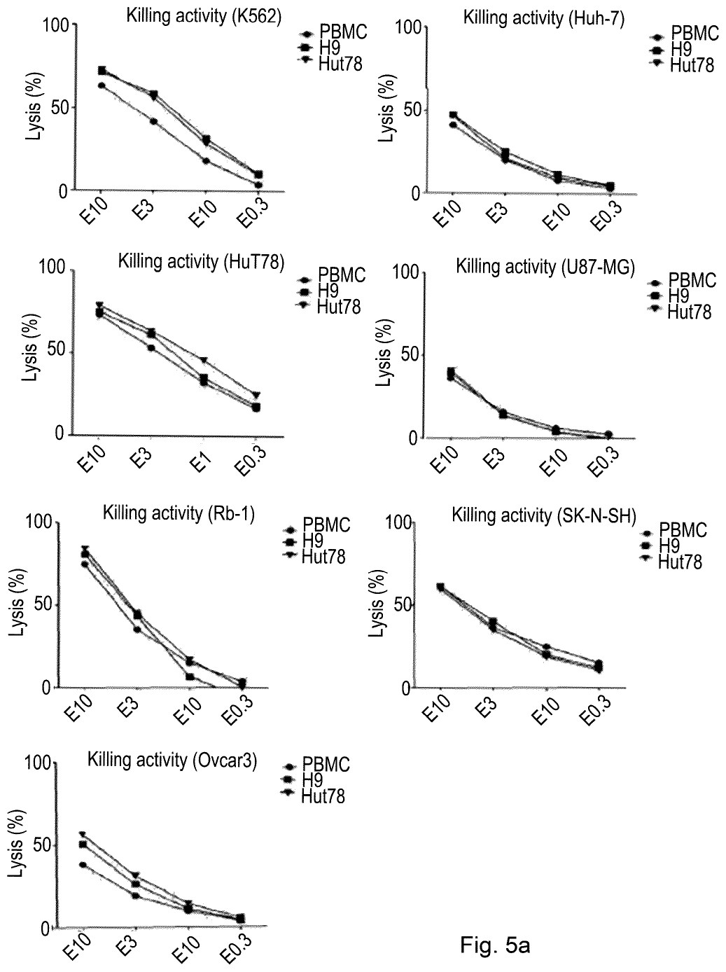

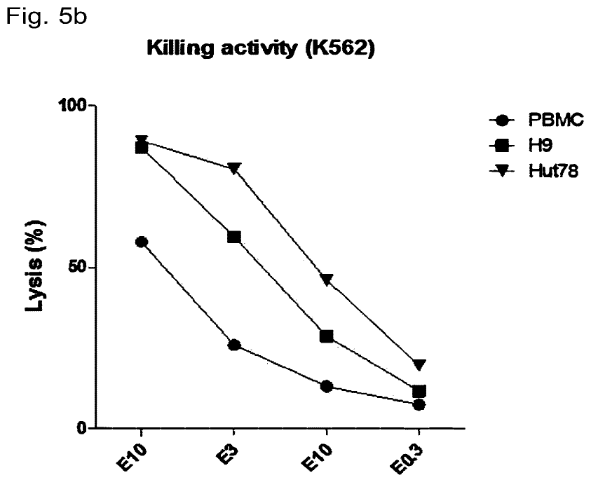

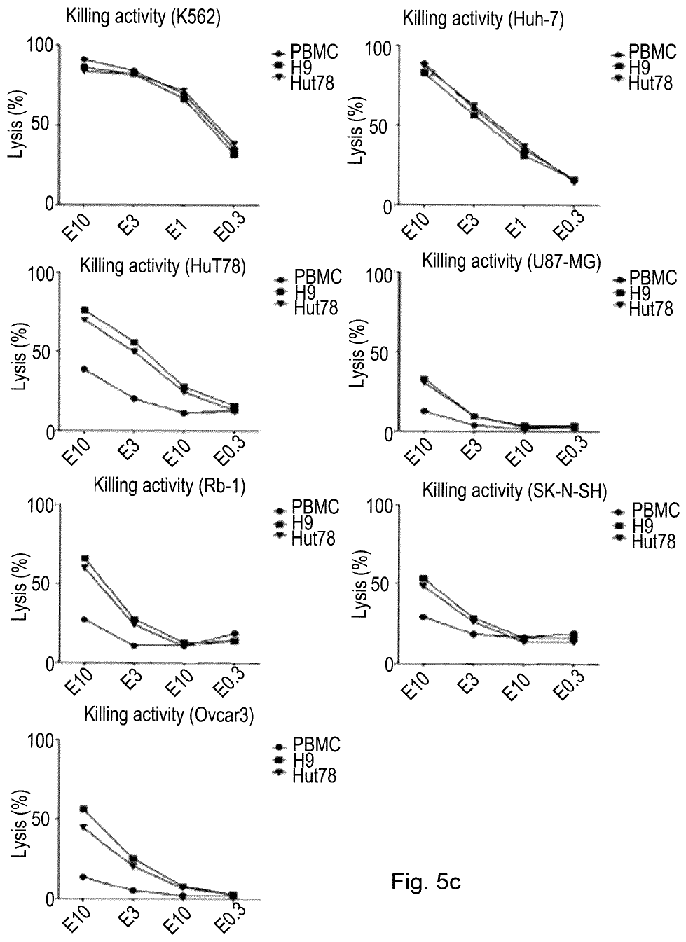

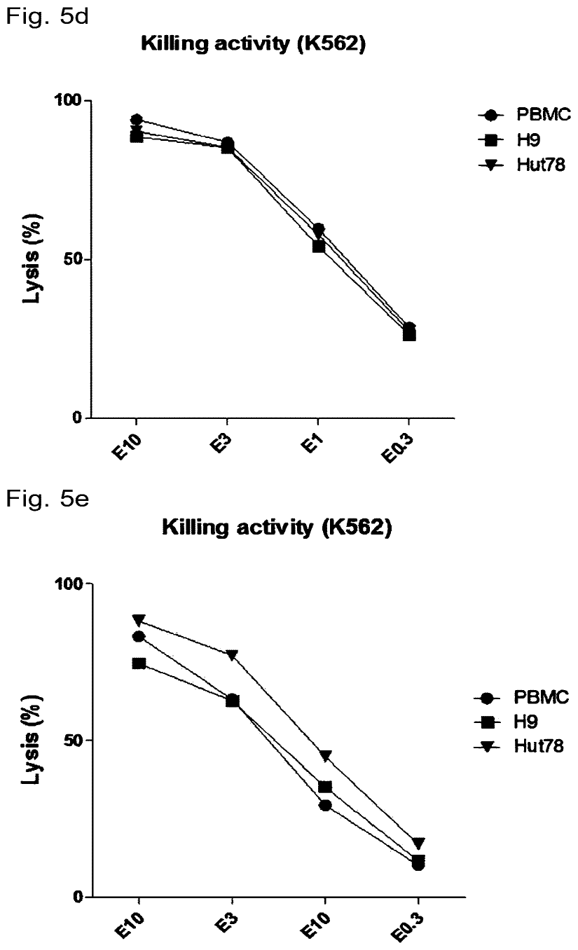

The cell killing activity of natural killer cells cultured under condition 1 (14-day culture of PBMC seed cells with various feeder cells) shown in Table 1 was evaluated against the blood cancer cell line K562, the liver cancer cell line HuH-7, the lymphoma cell line HuT78, the brain tumor cell line U87-MG, the retinoblastoma cell line SNUOT-Rbl, the neuroblastoma cell line SK-N-SH and the ovarian cancer cell line OVCAR-3. As a result, it was shown that culture with PBMC feeder cells showed a higher cell killing activity against all the tumor targets compared with culture with H9 and HuT78 (FIG. 5a).

The cell killing activity of natural killer cells cultured under condition 2 (21-day culture of PBMC seed cells) shown in Table 1 was measured. As a result, it was shown that the cell killing activity against the K562 tumor cell line was higher in the order of HuT78>H9>PBMCs (FIG. 5b).

In addition, the cell killing activity of natural killer cells cultured under condition 3 (14-day culture of CD3 (−) seed cells with various feeder cells) was evaluated against the blood cancer cell line K562, the liver cancer cell line HuH-7, the lymphoma cell line HuT78, the brain tumor cell line U87-MG, the retinoblastoma cell line SNUOT-Rbl, the neuroblastoma cell line SK-N-SH and the ovarian cancer cell line OVCAR-3. As a result, it was shown that culture with H9 and HuT78 showed a higher cell killing activity against most of the tumor targets (excluding K562) compared to culture with PBMC feeder cells, and particularly, this difference was more significant in the case of cancer cells having resistance to the killing activity of natural killer cells (FIG. 5c).

The cell killing activity of natural killer cells cultured under condition 4 (21-day culture of CD3 (−) seed cells) shown in Table 1 was measured. As a result, it was shown that the cell killing activity against the K562 tumor cell line was similar between the types of feeder cells (FIG. 5d). This insignificant difference in specificity between the types of feeder cells appears to be because PBMCs were used in the first stimulation of seed cells in the same manner and PBMCs and various T cells were used only in the second stimulation.

The cell killing activity of natural killer cells in the case of poorly proliferating donor cells, cultured under condition 3 of Table 3, was measured. As a result, it was shown that HuT78 showed the highest killing activity against the K562 tumor cell line and that H9 and PBMC feeder cells showed similar killing activities (FIG. 5e).

In conclusion, HuT78 showed the highest cell killing activity against various tumor cell lines under most of the conditions. Particularly, natural killer cells cultured with H9 and HuT78 showed higher killing activities against tumor cell lines having resistance, indicating that H9 and HuT78 cells are feeder cells which are better than PBMCs in terms of efficacy.

The characteristics of natural killer cells cultured under conditions 1, 2, 3 and 4 are summarized in the following Tables 2 to 5.

TABLE 2
Characteristics of natural killer cells cultured under condition 1

Mean(SD)

| | | | | | % of Cytotoxidity |
|---|---|---|---|---|---|
| | Expansion | Expansion | | % of | against K562 |
| | fold TNC | fold NK | Viability | CD3−CD56+ | ET ratio = 3:1 |
| PBMC | 147(62) | 272(180) | 77%(0) | 15.9%(12.5) | 41.5%(13.5) |
| H9 | 298(108) | 2752(386) | 83%(2) | 73.3%(7.0) | 58.7%(5.9) |
| HuT78 | 485(31) | 5649(2966) | 87%(1) | 83.3%(0.5) | 56.2%(3.9) |

TABLE 3
Characteristics of natural killer cells cultured under condition 2

Mean(SD)

| | | | | | % of Cytotoxidity |
|---|---|---|---|---|---|
| | Expansion | Expansion | | % of | against K562 |
| | fold TNC | fold NK | Viability | CD3−CD56+ | ET ratio = 3:1 |
| PBMC | 334(183) | 1257(1498) | 76%(2) | 17.4%(9.1) | 26.0%(8.7) |
| H9 | 358(60) | 2677(268) | 72%(3) | 61.1%(12.9) | 59.4%(11.8) |
| HuT78 | 2282(509) | 29455(21088) | 87%(5) | 83.5%(2.8) | 80.3%(6.2) |

TABLE 4
Characteristics of natural killer cells cultured under condition 3

Mean(SD)

| | | | | % of Cytotoxidity |
|---|---|---|---|---|
| | Expansion | | % of | against K562 |
| | fold TNC | Viability | CD3−CD56+ | ET ratio = 3:1 |
| PBMC | 184(81) | 92%(2) | 99.3%(0.4) | 84.2%(4.0) |
| H9 | 62(24) | 90%(1) | 99.5%(0.4) | 81.9%(2.8) |
| HuT78 | 217(1) | 91%(2) | 99.8%(1) | 81.9%(2.8) |

TABLE 5
Characteristics of natural killer cells cultured under condition 4

Mean(SD)

| | | | | % of Cytotoxidity |
|---|---|---|---|---|
| | Expansion | | % of | against K562 |
| | fold TNC | Viability | CD3−CD56+ | ET ratio = 3:1 |
| PBMC | 1402(506) | 91%(4) | 99.5%(0.3) | 87.0%(2.4) |
| H9 | 720(288) | 88%(4) | 99.6%(0.3) | 85.2%(6.0) |
| HuT78 | 1393(161) | 91%(5) | 99.8%(0.1) | 85.3%(4.1) |

### INDUSTRIAL APPLICABILITY

As described above, the method for producing natural killer cells using T cells according to the present invention is a method capable of producing natural killer cells by selectively proliferating only natural killer cells from a small amount of seed cells while maintaining the high killing activity of the natural killer cells. The method of the present invention can produce a large amount of natural killer cells that can be frozen, and thus is useful for commercialization of cell therapeutic agents.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1.-23. (canceled)

24. A method for producing a population of natural killer cells, the method comprising:
(a) providing seed cells comprising natural killer cells; and
(b) expanding the natural killer cells by culturing the seed cells with a plurality of cells from an inactivated CD4(+) T cell line to produce expanded natural killer cells, thereby producing the population of natural killer cells.

25. The method of claim 24, wherein the seed cells of step (a) are CD3(+)-depleted seed cells.

26. The method of claim 24, wherein the seed cells provided in step (a) have not been expanded ex vivo or in vitro.

27. The method of claim 24, wherein the seed cells provided in step (a) comprise mononuclear cells comprising natural killer cells.

28. The method of claim 24, wherein the seed cells provided in step (a) are selected from peripheral blood cells, peripheral blood leukocytes, and PBMCs (peripheral blood mononuclear cells).

29. The method of claim 24, wherein the seed cells provided in step (a) are selected from enriched natural killer cells and isolated natural killer cells.

30. The method of claim 24, wherein the cells from an inactivated CD4(+) T cell line are selected from the group consisting of H9, HuT78, Loucy, Molt3, PEER, and combinations thereof.

31. The method of claim 24, wherein the cells from an inactivated CD4(+) T cell line are selected from the group consisting of H9, HuT78, and combinations thereof.

32. The method of claim 24, wherein the cells from an inactivated CD4(+) T cell line are H9 cells.

31. The method of claim 24, wherein the cells from an inactivated CD4(+) T cell line are HuT78 cells.

34. The method of claim 24, wherein the culturing is carried out in a medium comprising a T-cell stimulating antibody and an interleukin protein.

35. The method of claim 34, wherein the T-cell stimulating antibody is OKT3, UCHT1, HT3a, or a combination thereof.

36. The method of claim 35, wherein the T-cell stimulating antibody is OKT3.

37. The method of claim 34, wherein the interleukin protein is IL-2, IL-12, IL-15, IL18, IL21, or a combination thereof.

38. The method of claim 37, wherein the interleukin protein is IL-2.

39. The method of claim 24, wherein the culturing is carried out for 5-60 days.

40. The method of claim 24, further comprising:
(c) culturing the expanded natural killer cell(s) produced in step (b) with a second plurality of cells from an inactivated CD4(+) T cell line.

41. Natural killer cell(s) produced by the method of claim 24.

42. A pharmaceutical composition comprising the natural killer cell(s) of claim 41 and a pharmaceutically acceptable carrier.

43. A method for preventing or treating cancer or infectious disease, the method comprising:
administering to a subject in need thereof a therapeutically effective amount of a population of natural killer cells produced by the method of claim 24.
